(19) United States
(12) Patent Application Publication
(10) Pub. No.: US 2009/0208472 A1
(43) Pub. Date: Aug. 20, 2009

(54) OIL-IN-WATER EMULSION COMPOSITION CONTAINING LICORICE-DERIVED POLYPHENOL

(75) Inventors: Midori Sakai, Hyogo (JP); Toshinori Ikehara, Hyogo (JP)

Correspondence Address: SUGHRUE MION, PLLC, 2100 PENNSYLVANIA AVENUE, N.W., SUITE 800, WASHINGTON, DC, 20037, US

(73) Assignee: KANEKA CORPORATION, Osaka-shi (JP)

(21) Appl. No.: 12/280,453

(22) PCT Filed: Feb. 23, 2007
(86) PCT No.: PCT/JP2007/053368

§ 371 Date: Jan. 30, 2009

(30) Foreign Application Priority Data
Feb. 24, 2006 (JP) 2006-048362
Jul. 21, 2006 (JP) 2006-198909

(51) Int. Cl. A61K 31/22; A61K 31/122

(52) U.S. Cl. 424/94.1; 514/546

(57) ABSTRACT

The present invention provides a licorice polyphenol-containing oil-in-water emulsion composition comprising medium-chain triglyceride, a licorice hydrophobic extract comprising, as a main component, licorice polyphenol in a weight ratio relative to the medium-chain triglyceride of 1-50%, and a polyglycerol fatty acid ester comprised of a fatty acid residue having a carbon number of not less than 14. Since the composition of the present invention is superior in transparency, stable to acid and heat, soluble in water, it can be used for food, drink, pharmaceutical product, quasi-drug, cosmetic and the like, which enables efficient supply of a licorice hydrophobic extract such as licorice polyphenol and the like.

### TECHNICAL FIELD OF THE INVENTION

The present invention relates to an aqueous composition containing a licorice hydrophobic extract, which can be used for foods and drinks such as health food, Food with health claims (Food for specified health uses, Food with nutrient function claims) and the like, pharmaceutical products, quasi-drugs, cosmetics and the like.

### BACKGROUND OF THE INVENTION

Licorice is known as a crude drug, and since glycyrrhizin (glycyrrhizic acid), which is the main component of a water extract of licorice, has superior pharmacological actions such as an anti-inflammatory action, an antitumor action, an antiallergic action and the like, it has been widely utilized for foods, pharmaceutical products, cosmetics and the like. In addition, since glycyrrhizin is about 200 times as sweet as sucrose, it is also used as a sweetener.

On the other hand, a licorice hydrophobic component extracted from licorice or a licorice water extract residue with an organic solvent and the like has been confirmed to show many useful actions such as an antioxidant action, an antibacterial action, an enzyme inhibitory action, an antitumor action, an antiallergic action, an antiviral action and the like. However, the licorice hydrophobic component is difficult to use because it hardly dissolves in water and general oil, and sometimes becomes unstable in the state of an extract because it is easily consolidated and easily colored and the like. Therefore, the problems have been solved by a method including mixing a licorice hydrophobic extract with a fat-and-oil solvent containing an liposoluble polyvalent alcohol fatty acid ester by not less than 10% (patent reference 1). Nevertheless, the fat-and-oil composition containing the licorice hydrophobicity component does not aim at direct dissolution in an aqueous target product and the like.

Patent reference 2 discloses processing, in the presence of an emulsifier, licorice hydrophobic flavonoid, which has been dissolved in medium-chain triglyceride, to give an oil-in-water emulsion. However, it merely aims at water-dispersibility and does not aim at transparency or acid resistance.

Patent reference 3 suggests a composition containing a licorice oil extract and polyglycerol lauric acid ester.

Patent reference 4 discloses a concentrated emulsion containing a fat-and-oil composition containing a licorice hydrophobic component and glycerol fatty acid ester as an emulsifier. However, the concentrated emulsion is an emulsion having no transparency and considered unsuitable for a use requiring transparency.

Patent reference 1: WO03/084556

Patent reference 2: JP-A-2-204417

Patent reference 3: JP-A-2003-176233

Patent reference 4: WO2005/011672

### DISCLOSURE OF THE INVENTION

### Problems to be Solved by the Invention

As mentioned above, application of conventional licorice hydrophobic component-containing fat-and-oil compositions is limited for aqueous targets since they lack dispersibility and transparency, have insufficient heat resistance and insufficient acid resistance, and the like. The object of the present invention is to provide an aqueous composition of a licorice hydrophobic extract, which maintains the state of an oil-in-water emulsion, enables efficient supply and has high bioavailability. In addition, the present invention aims to maintain high transparency of an aqueous target without impairing the inherent properties thereof even when a licorice hydrophobic component is added to the target.

### Means of Solving the Problems

The present inventors have conducted intensive studies in view of the above-mentioned situation and found that a particular component constitution provides an effect of preventing insolubilization of a licorice hydrophobic component, and that a stable aqueous composition can be obtained by producing an aqueous composition using same, which resulted in the completion of the present invention.

Accordingly, the present invention provides the following.

(1) A licorice polyphenol-containing oil-in-water emulsion composition comprising medium-chain triglyceride, a licorice hydrophobic extract comprising, as a main component, licorice polyphenol in a weight ratio relative to the medium-chain triglyceride of 1-50%, and a polyglycerol fatty acid ester comprised of a fatty acid residue having a carbon number of not less than 14, namely, a licorice polyphenol-containing oil-in-water emulsion composition comprising a licorice hydrophobic extract comprising licorice polyphenol as a main component, medium-chain triglyceride, and a polyglycerol fatty acid ester comprised of a fatty acid residue having a carbon number of not less than 14, wherein the weight ratio of the licorice polyphenol to the medium-chain triglyceride is 1-50%.
(2) The oil-in-water emulsion composition of (1), wherein the weight ratio of the total weight of the licorice hydrophobic extract and the medium-chain triglyceride to the polyglycerol fatty acid ester is within the range of 10:1-1:10.
(3) The oil-in-water emulsion composition of (1) or (2), wherein the degree of polymerization of polyglycerol in the polyglycerol fatty acid ester is not less than 6.
(4) The oil-in-water emulsion composition of any one of (1) to (3), wherein the degree of polymerization of polyglycerol in the polyglycerol fatty acid ester is 10, and the fatty acid residue is at least one kind selected from the group consisting of myristic acid, palmitic acid, stearic acid and oleic acid.
(5) The oil-in-water emulsion composition of any one of (1) to (4), further comprising coenzyme Q10.
(6) The oil-in-water emulsion composition of any one of (1) to (5), having a median size of emulsion particles of not more than 100 nm.
(7) The oil-in-water emulsion composition of (6), which is obtained by a homogenization pressure treatment at not less than 50 MPa.
(8) A dry powder obtained by adding an excipient to the oil-in-water emulsion composition of any one of (1) to (7), and removing water from the mixture.
(9) A food, drink, pharmaceutical product, quasi-drug, cosmetic or feed for animal, comprising the oil-in-water emulsion composition of any one of (1) to (7), or the dry powder of (8)
(10) The drink of (9), wherein the content of licorice polyphenol is 10-200 mg per a package unit.
(11) The drink of (9), wherein the content of glabridin is 1-20 mg per a package unit.

### EFFECT OF THE INVENTION

The present invention can provide a stable oil-in-water emulsion composition containing a hardly water-soluble licorice hydrophobic component. Since the oil-in-water emulsion composition of the present invention is water-soluble, an aqueous target product having high bioavailability (food, drink, pharmaceutical product, quasi-drug, cosmetic, feed for animal etc.), which enables efficient supply of a licorice hydrophobic component, can be produced using the composition. In addition, since the oil-in-water emulsion composition of the present invention is superior in acid resistance and heat resistance, it can be applied to various uses.

### BEST MODE FOR CARRYING OUT THE INVENTION

The present invention is explained in detail in the following.

The oil-in-water emulsion composition containing licorice polyphenol of the present invention (sometimes to be referred to as the oil-in-water emulsion composition of the present invention) is a licorice polyphenol-containing oil-in-water emulsion composition comprising a licorice hydrophobic extract comprising licorice polyphenol as a main component, medium-chain triglyceride, and a polyglycerol fatty acid ester comprised of a fatty acid residue having a carbon number of not less than 14, wherein the weight ratio of the licorice polyphenol to the medium-chain triglyceride is 1-50%.

The oil-in-water emulsion composition containing licorice polyphenol of the present invention comprises a licorice hydrophobic extract comprising licorice polyphenol as a main component, medium-chain triglyceride, a polyglycerol fatty acid ester comprised of a fatty acid residue having a carbon number of not less than 14, and an aqueous phase component.

In the present invention, examples of the licorice to be the origin of the licorice hydrophobic extract include plants of the Leguminosae Glycyrrhiza genus, such as Glycyrrhiza glabra (G. glabra), Glycyrrhiza uralensis (G. uralensis), Glycyrrhiza inflata (G. inflata), Glycyrrhiza eurycarpa (G. eurycarpa), Glycyrrhiza aspera (G. aspera) and the like, preferably, G. glabra, G. uralensis and G. inflata, and particularly preferably G. glabra.

The licorice hydrophobic extract to be used in the present invention is a licorice extract containing licorice polyphenol as a main component. Herein, the “as a main component” means that, for example, not less than 50%, preferably not less than 60%, of the licorice hydrophobic extract is a polyphenol component. The method for obtaining the licorice hydrophobic extract is not particularly limited, and may be a known method. For example, the extract can be obtained by extracting the above-mentioned licorice or a powder thereof, or an aqueous extract residue with an organic solvent (e.g., ethanol, acetone, ethyl acetate etc.). Furthermore, since the oil-in-water emulsion composition of the present invention employs medium-chain triglyceride as a fat-and-oil component, it is also preferable to obtain a licorice hydrophobic extract as a solution of medium-chain triglyceride according to the methods described in WO2003/084556 (patent reference 1) and WO2005/011672 (patent reference 4). To be specific, a medium-chain triglyceride solution of a licorice hydrophobic extract can be prepared by extracting a licorice rhizome with ethanol, adding medium-chain triglyceride to an ethanol solution containing the obtained licorice hydrophobic extract and removing ethanol.

The licorice hydrophobic extract of the present invention contains, as a licorice polyphenol component, at least one kind of a compound from glycycoumarin, glycyrol, glycyrin, liquiritigenin, glicoricone, glabridin, glabrene, glabrol, 3′-hydroxy-4′-O-methylglabridin, 4′-O-methylglabridin, glyurallin B, licocoumarone, gancaonin I, dehydroglyasperin D, echinatin, isolicoflavonol, dehydroglyasperin C, glyasperin B, glycyrrhisoflavanone, lupiwighteone, glyasperin D, semilicoisoflavone B and the like. Among these, glabridin, glabrene, glabrol, 3′-hydroxy-4′-O-methylglabridin and 4′-O-methylglabridin are preferable.

The oil-in-water emulsion composition of the present invention needs to have a licorice polyphenol content within the range of 1-50% in the weight ratio relative to medium-chain triglyceride. In this context, the licorice polyphenol component can be quantified on the basis of glabridin by the method described in the below-mentioned Examples.

The medium-chain triglyceride to be used in the present invention is not particularly limited as long as it is comprised of fatty acid having a carbon number of 6 to 12. Preferred is triglyceride comprised of saturated fatty acid having a carbon number of 8 or 10, and most preferred is one containing triglyceride comprised of saturated fatty acid having a carbon number of 8 as a main component.

In the oil-in-water emulsion composition of the present invention, polyglycerol fatty acid ester comprised of a fatty acid residue having a carbon number of not less than 14 is used as an emulsifier. The fatty acid residue having a carbon number of not less than 14 is not particularly limited, and a polyglycerol fatty acid ester having saturated or unsaturated fatty acid having a carbon number of 14-18 as a fatty acid residue such as myristic acid, palmitic acid, stearic acid, oleic acid and the like is preferably used. Needless to say, a mixture thereof can also be used. While the esterification degree of polyglycerol fatty acid ester is not particularly limited, polyglycerol monofatty acid ester (e.g., polyglycerol monomyristic acid ester, polyglycerol monopalmitic acid ester, polyglycerol monostearic acid ester, polyglycerol monooleic acid ester) and the like can be mentioned. The polyglycerol fatty acid ester to be used in the present invention is preferably hydrophilic, and HLB thereof is preferably not less than 8, more preferably not less than 10, more preferably 12 or more. While the average polymerization degree of glycerol in polyglycerol fatty acid ester is not particularly limited, it is preferably not less than 6, more preferably not less than 10, and most preferred is a decaglycerol fatty acid ester having an average polymerization degree of 10.

While the amount of use of polyglycerol fatty acid ester in the composition of the present invention is not particularly limited, the weight ratio of the total amount of a licorice hydrophobic extract and medium-chain triglyceride, to polyglycerol fatty acid ester is preferably within the range of 10:1-1:10. That is, polyglycerol fatty acid ester is preferably used within the range of 10-1000 parts by weight, more preferably 50-500 parts by weight, relative to 100 parts by weight of the total amount of a licorice hydrophobic extract and medium-chain triglyceride. When the amount of the aforementioned polyglycerol fatty acid ester to be added is smaller than 50 parts by weight, a stable aqueous composition cannot be achieved.

As the aqueous phase component of the oil-in-water emulsion composition of the present invention, water, polyvalent alcohol, and a mixture thereof can be used. Examples of the above-mentioned polyvalent alcohol include saccharides such as liquid sugar syrup and the like, and sugar alcohol such as glycerol, sorbitol and the like. From the aspect of emulsion property, it is preferable to use water alone or use water as a main component of an aqueous phase component. The safety from the aspect of hygiene during a long-term preservation is improved by increasing the amount of polyvalent alcohol as an aqueous phase component. The content of the aqueous phase component in the oil-in-water emulsion composition of the present invention is generally about 50-99 wt % of the total weight of the oil-in-water emulsion composition.

The oil-in-water emulsion composition containing licorice polyphenol of the present invention may concurrently contain a fat-and-oil component other than medium-chain triglyceride. In this case, the fat-and-oil component is not particularly limited as long as it does not affect the emulsion and, for example, vegetable oil such as corn oil, rapeseed oil, high erucic acid rapeseed oil, soybean oil, olive oil, safflower oil, cottonseed oil, sunflower oil, rice bran oil, Japanese basil oil, perilla oil, flaxseed oil, evening primrose oil, cacao butter, peanut oil, palm oil, palm kernel oil and the like, animal oils such as fish oil, beef fat, lard, milk fat, egg yolk oil and the like, fats and oils obtained by fractionation, hydrogenation, transesterification and the like of the above as starting materials, or a mixed oil thereof can be used.

The oil-in-water emulsion composition containing licorice polyphenol of the present invention can further contain other liposoluble physiologically active substance. Examples of such liposoluble physiologically active substance include liposoluble vitamins such as vitamins A, D, E, K, P and the like, coenzyme Q (ubiquinone and ubiquinol), lipoic acid, L-carnitine and the like. Among these, coenzyme Q, particularly coenzyme Q10, is preferably contained. Coenzyme Q10 is a liposoluble substance essential for the functional maintenance of living organisms, which is known to be localized in mitochondria, lysosome, Golgi body, microsome, peroxisome, cellular membrane and the like, and involved, as a constituent component of an electron transport system, in ATP production activation, in vivo antioxidant action and membrane-stabilization. It has recently been drawing attention as a heath material, and often used not only in the field of pharmaceutical products, but also health food, Food with health claims and the like. The oil-in-water emulsion composition containing licorice polyphenol of the present invention is superior in that it can not only stably formulate a preparation of a licorice hydrophobic extract, but also a preparation of a liposoluble physiologically active component, such as coenzyme Q10, stably to afford a highly transparent aqueous composition. The amount of the liposoluble physiologically active substance to be contained in the oil-in-water emulsion composition containing licorice polyphenol of the present invention is not particularly limited, and appropriately selected from the range that does not impair emulsifiability and stability of the preparation. For example, in the case of coenzyme Q10, the content ratio of licorice polyphenol and coenzyme Q10 is preferably selected from the range of 1:10-10:1, from the aspects of stability and effect and efficacy of the preparation. In addition to the above, liposoluble components such as liposoluble flavor, essential oil, coloring agent, antioxidant, specific gravity adjuster and the like, and aqueous components such as aqueous vitamins (e.g., vitamin C and the like), organic acid, amino acids (e.g., L-carnitine and the like), various salts as taste component and the like can be contained in the oil-in-water emulsion composition containing licorice polyphenol of the present invention as long as they do not influence the emulsification.

The emulsification method to obtain the oil-in-water emulsion composition containing licorice polyphenol in the present invention is not particularly limited, and a mechanical emulsification method using a general emulsification machine can be mentioned. As the apparatus to be used for a mechanical emulsification method, high-speed stirring emulsification machines such as TK homomixer (manufactured by Primix Corporation), Filmics (manufactured by Primix Corporation), Polytron (manufactured by KINEMATICA AG), Hiscotron (manufactured by microtec nition), Cleamix W-Motion (manufactured by M Technique Corporation) and the like, high-pressure emulsification machines such as microfluidizer (manufactured by Mizuho Industrial Co., Ltd.), Ultimizer system (manufactured by Sugino Machine Limited), nanomizer (manufactured by Yoshida Kikai Co., Ltd.), Manton-Gaulin homogenizer and the like, colloid mill, ultrasonication homogenizer and the like can be mentioned. Besides the mechanical emulsification methods, membrane emulsification method, microchannel emulsification method, natural emulsification method, phase inversion emulsification method, gel emulsification method, D phase emulsification method and the like can also be utilized.

By the aforementioned emulsification method, the oil-in-water emulsion composition of the present invention is adjusted to have an emulsion particle median size of preferably not more than 100 nm, more preferably not more than 80 nm, still more preferably not more than 50 nm. In the oil-in-water emulsion composition of the present invention, the median size of the emulsion particles is generally not less than about 10 nm. The median size of emulsion particles is used as an index showing the stability of an emulsion. The median size of emulsion particles in an emulsion showing a uniform dispersion state is generally not more than 100 μm (100000 nm). What is called a normal emulsion is a cloudy solution having a median size within the range of 0.1-100 μm (100-100000 nm). When the median size is not more than 100 nm, the emulsion becomes an almost transparent solution, and when it is less than 50 nm, it becomes a highly transparent solution. In the present invention, the oil-in-water emulsion composition is adjusted to have an emulsion particle median size of not more than 100 nm, whereby a transparent solution can be obtained, while improving the preservation stability, acid resistance and heat resistance.

To achieve a desired median size, a high-pressure emulsification machine is preferably used from among the aforementioned emulsification methods. When a high-pressure emulsification machine is used, the homogenization pressure is not less than 20 MPa, preferably not less than 50 MPa, more preferably not less than 100 MPa. When it is less than 20 MPa, the median size may not be 100 nm or below. Since highly sharp particle size distribution affords good preservation stability, a high-pressure emulsification treatment (homogenization pressure treatment) is preferably performed 10 times or more. Moreover, other emulsification method may be employed as long as it affords a shear force equivalent to that of a high-pressure emulsification machine. Examples thereof include the aforementioned Cleamix W-Motion and Filmics, using which the median size can be adjusted to 100 nm or below by appropriately setting the stirring rate and stirring time.

One example of the production methods of the oil-in-water emulsion composition containing licorice polyphenol of the present invention is explained in the following. A medium-chain triglyceride solution containing a predetermined amount of a licorice hydrophobic extract, polyglycerol fatty acid ester, and other optional component (liposoluble component) are dissolved by stirring in a general turbine type stirrer, a paddle type stirrer and the like while heating at a predetermined temperature to give an oil phase. The polyglycerol fatty acid ester may be added to an aqueous phase. Then, the aqueous phase is added to the adjusted oil phase with stirring to perform a preliminary emulsification. The obtained preliminarily emulsified solution is subjected to an emulsification treatment in a high-pressure emulsification machine to afford a desired particle size. While the temperature of the emulsification treatment is not particularly limited as long as the polyglycerol fatty acid ester is dissolved in water, it is preferably within the range of 50-80° C.

The oil-in-water emulsion composition containing licorice polyphenol of the present invention can be directly ingested or ingested after filling the composition in a capsule. Furthermore, the composition can be used, for example, for drinks such as milk beverage, beverage, energy drink, beauty drink and the like, confectionery such as chewing gum, chocolate, candy, jelly, biscuit, cracker and the like, frozen desserts such as ice cream, ice desserts and the like, noodles such as Japanese wheat noodles, Chinese noodles, spaghetti, instant noodles and the like, processed seafood paste products such as fish cake, tube-like fish sausage, square-shaped fish cake and the like, seasoning such as dressing, mayonnaise, sauce and the like, various retort foods such as bread, ham, soup, various pouch-packed food, various frozen foods and the like by directly adding to food and the like or preparing by dissolving in water. Moreover, the composition can also be used, for example, for pet food, livestock feeds and the like. Since the composition is particularly superior in aqueous transparency, acid resistance and heat resistance, it is preferably used for drinks such as beverage, energy drink, beauty drink and the like. In the present invention, the food and drink include health food, Food with health claims (Food for specified health uses, Foods with nutrient function claims) and the like. The oil-in-water emulsion composition containing licorice polyphenol of the present invention can be also used for pharmaceutical product, quasi-drug and cosmetic.

In addition, the oil-in-water emulsion composition containing licorice polyphenol of the present invention can also be processed into a dry powder by adding saccharides such as dextrin, lactose and the like, sugar alcohols such as erythritol and the like, excipients such as gum arabic, gum ghatti and the like, and removing water by a method known per se such as spray dry, freeze-dry and the like. While the amount of the excipient to be added is not particularly limited, it is generally about 50-90 wt % relative to the total weight of a dry powder. Using sugar alcohols as an excipient, the content of the licorice hydrophobic extract in a dry powder can be increased. For example, when sugar alcohol is used in combination as an excipient, the amount ratio of a licorice hydrophobic extract/excipient can be increased, as compared to the use of gum arabic alone. The obtained dry powder can be easily processed into an aqueous solution containing a licorice hydrophobic component by dissolution thereof in water. The dry powder and an aqueous solution thereof can be ingested as they are. However, by addition to food and the like, they can be used for food, drinks, animal feed and the like such as those exemplified as the aforementioned oil-in-water emulsion composition containing licorice polyphenol of the present invention. The dry powder can also be used as a pharmaceutical product, a quasi-drug or a cosmetic.

The amount of the oil-in-water emulsion composition containing licorice polyphenol of the present invention and the aforementioned dry powder to be added to food and the like is not particularly limited. For drinks, for example, the content of licorice polyphenol is about 10-200 mg, and the content of glabridin is about 1-20 mg, both per one package unit (e.g., 1 drink).

### EXAMPLES

The present invention is explained in more detail in the following by referring to Examples, which are not to be construed as limitative.

### <Median Size of Emulsion and Aqueous Composition>

The median size of the oil-in-water emulsion compositions and aqueous compositions of Examples and Comparative Examples was measured by LB-550 (manufactured by Horiba, Ltd.).

### <Evaluation of Stability of Emulsion and Aqueous Compositions Against Acid and Heat>

Water or aqueous citric acid solution at pH 3 (indicated as “pH3 water” in Tables) was added to oil-in-water emulsion compositions or aqueous compositions of Examples and Comparative Examples in an amount of 5000 parts by weight per 1 part by weight of the total amount of licorice polyphenol and medium-chain triglyceride, and the median size was measured using LB-550 after the addition and after heating the mixture at 75° C. for 15 min.

### Preparation Example 1

Rhizome (1.0 Kg) of licorice (G. glabra) from Afghan was extracted (45° C., 2 hr, twice) with ethanol (5.0 L) and concentrated under reduced pressure to give a concentrated liquid (0.45 L). Then, the concentrated liquid (0.3 L) was further concentrated and treated with activated carbon to give a licorice hydrophobic extract-containing ethanol solution (123.6 g, containing 24.8 g of licorice hydrophobic extract).

### Preparation Example 2

The licorice hydrophobic extract-containing ethanol solution (63.9 g) obtained in Preparation Example 1 and medium-chain triglyceride (Actor M2; Riken Vitamin Co., Ltd., fatty acid composition C8:C10=99:1, 18.8 g) were mixed. The mixture was stirred for 1 hr during heating about at 80° C. and concentrated under reduced pressure to remove ethanol. 28.7 g obtained by concentration under reduced pressure was suction filtered to remove an insoluble material, which was washed with hexane. The resulting recovered oil was added to the earlier filtrate. Medium-chain triglyceride (Actor M2, 4.5 g) was added to the recovered filtrate (26.2 g) to give a licorice hydrophobic extract-containing medium-chain triglyceride solution (30.7 g, containing 8.9 g of licorice hydrophobic extract).

### HPLC Analysis

### <Preparation of HPLC Analysis Sample>

The above-mentioned licorice hydrophobic extract-containing medium-chain triglyceride solution (1 g) was dissolved in methanol for HPLC, and the total amount was adjusted to 100 ml.

### <HPLC Conditions>

column: YMC, J'sphere ODS-H80, 4.6×250 mm
column temperature: 40° C.
mobile phase: A=20 mM aqueous phosphoric acid solution
B=acetonitrile:methanol (50:50=v/v)
   gradient: conditions under which the ratio of B relative to mobile phase A was maintained constant at 50% for 20 minutes from the start of the analysis, raised at a given rate to reach 80% in 75 minutes after the 20 minutes, maintained constant at 100% from 75 minutes to 80 minutes and maintained constant at 50% from 80 minutes to 100 minutes
   flow rate: 1 ml/min
   wavelength: UV 282 nm
   sample injection volume: 20 μL

### <Analysis Results>

The content of each component in 1 g of the licorice hydrophobic extract-containing medium-chain triglyceride solution was glabrene (4.4 mg), glabridin (30.0 mg), glabrol (6.0 mg), and 4′-O-methylglabridin (5.2 mg).

### Polyphenol Analysis

As a result of the polyphenol content (flavonoid component content) measurement by Folin-Denis assay and using glabridin (manufactured by Wako Pure Chemical Industries, Ltd.) as a standard substance, the total content of polyphenol in 1 g of the licorice hydrophobic extract-containing medium-chain triglyceride solution was 239.1 mg.

### Example 1

An oil-in-water emulsion composition containing licorice polyphenol was prepared at the blending ratio shown in Table 1. Specifically, licorice hydrophobic extract-containing medium-chain triglyceride solution of Preparation Example 2 (1 part by weight) as an oil phase and decaglycerol monomyristic acid ester (manufactured by Mitsubishi-Kagaku Foods Corporation; M-7D, 5 parts by weight) as polyglycerol fatty acid ester were heated to 60° C. and dissolved completely. Similarly, as an aqueous phase, water (94 parts by weight) was heated to 60° C. Then, the aqueous phase was added to the oil phase with stirring, and the mixture was treated 10 times at an emulsification pressure of 100 MPa using Nanomizer II (manufactured by Yoshida Machinery Co. Ltd.) to give an oil-in-water emulsion composition containing licorice polyphenol. The particle size of the obtained oil-in-water emulsion composition containing licorice polyphenol was measured, and the results of the stability evaluation against acid and heat are summarized in Table 1.

### Example 2

An oil-in-water emulsion composition containing licorice polyphenol was prepared at the blending ratio shown in Table 1. The oil-in-water emulsion composition containing licorice polyphenol was prepared in the same manner as in Example 1 except that licorice hydrophobic extract-containing medium-chain triglyceride solution of Preparation Example 2 (1 part by weight) as an oil phase, decaglycerol monopalmitic acid ester (manufactured by Mitsubishi-Kagaku Foods Corporation; P-8DS, 5 parts by weight) as polyglycerol fatty acid ester and water (94 parts by weight) as an aqueous phase were used. The particle size of the obtained oil-in-water emulsion composition containing licorice polyphenol was measured, and the results of the stability evaluation against acid and heat are summarized in Table 1.

### Example 3

An oil-in-water emulsion composition containing licorice polyphenol was prepared at the blending ratio shown in Table 1. The oil-in-water emulsion composition containing licorice polyphenol was prepared in the same manner as in Example 1 except that licorice hydrophobic extract-containing medium-chain triglyceride solution of Preparation Example 2 (5 parts by weight) as an oil phase, decaglycerol monostearic acid ester (RIKEN VITAMIN CO., LTD.; J-0081, 10 parts by weight) as polyglycerol fatty acid ester and 85 parts by weight of an aqueous phase were used. The particle size of the obtained oil-in-water emulsion composition containing licorice polyphenol was measured, and the results of the stability evaluation against acid and heat are summarized in Table 1.

### Example 4

An oil-in-water emulsion composition containing licorice polyphenol was prepared at the blending ratio shown in Table 1. The oil-in-water emulsion composition containing licorice polyphenol was prepared in the same manner as in Example 1 except that licorice hydrophobic extract-containing medium-chain triglyceride solution of Preparation Example 2 (3 parts by weight) as an oil phase, decaglycerol monooleic acid ester (RIKEN VITAMIN CO., LTD.; J-0381, 10 parts by weight) as polyglycerol fatty acid ester and water (87 parts by weight) as an aqueous phase were used. The particle size of the obtained oil-in-water emulsion composition containing licorice polyphenol was measured, and the results of the stability evaluation against acid and heat are summarized in Table 1.

### Example 5

An oil-in-water emulsion composition containing licorice polyphenol was prepared at the blending ratio shown in Table 1. The oil-in-water emulsion composition containing licorice polyphenol was prepared in the same manner as in Example 1 except that licorice hydrophobic extract-containing medium-chain triglyceride solution of Preparation Example 2 (1 part by weight) as an oil phase, decaglycerol monomyristic acid ester (M-7D, 1 part by weight) as polyglycerol fatty acid ester and water (98 parts by weight) as an aqueous phase were used. The particle size of the obtained oil-in-water emulsion composition containing licorice polyphenol was measured, and the results of the stability evaluation against acid and heat are summarized in Table 1.

### Example 6

An oil-in-water emulsion composition containing licorice polyphenol was prepared at the blending ratio shown in Table 1. The oil-in-water emulsion composition containing licorice polyphenol was prepared in the same manner as in Example 1 except that licorice hydrophobic extract-containing medium-chain triglyceride solution of Preparation Example 2 (1 part by weight) as an oil phase, decaglycerol monostearic acid ester (J-0081, 2 parts by weight) as polyglycerol fatty acid ester and water (97 parts by weight) as an aqueous phase were used. The particle size of the obtained oil-in-water emulsion composition containing licorice polyphenol was measured, and the results of the stability evaluation against acid and heat are summarized in Table 1.

### Example 7

An oil-in-water emulsion composition containing licorice polyphenol was prepared at the blending ratio shown in Table 1. The oil-in-water emulsion composition containing licorice polyphenol was prepared in the same manner as in Example 1 except that licorice hydrophobic extract-containing medium-chain triglyceride solution of Preparation Example 2 (1 part by weight) as an oil phase, decaglycerol monooleic acid ester (Taiyo Kagaku Co., Ltd.; Q-17S, 10 parts by weight) as polyglycerol fatty acid ester and water (89 parts by weight) as an aqueous phase were used. The particle size of the obtained oil-in-water emulsion composition containing licorice polyphenol was measured, and the results of the stability evaluation against acid and heat are summarized in Table 1.

### Example 8

An oil-in-water emulsion composition containing licorice polyphenol was prepared at the blending ratio shown in Table 1. Licorice hydrophobic extract-containing medium-chain triglyceride solution of Preparation Example 2 (1 part by weight) as an oil phase, decaglycerol monomyristic acid ester (M-7D, 5 parts by weight) as polyglycerol fatty acid ester and water (94 parts by weight) as an aqueous phase were used. In addition, the oil-in-water emulsion composition containing licorice polyphenol was prepared in the same manner as in Example 1 except that the emulsification pressure was set to 50 MPa. The particle size of the obtained oil-in-water emulsion composition containing licorice polyphenol was measured, and the results of the stability evaluation against acid and heat are summarized in Table 1.

### Example 9

An oil-in-water emulsion composition containing licorice polyphenol was prepared at the blending ratio shown in Table 1. Licorice hydrophobic extract-containing medium-chain triglyceride solution of Preparation Example 2 (1 part by weight) as an oil phase, decaglycerol monomyristic acid ester (M-7D, 5 parts by weight) as polyglycerol fatty acid ester and water (94 parts by weight) as an aqueous phase were used. In addition, the oil-in-water emulsion composition containing licorice polyphenol was prepared in the same manner as in Example 1 except that the emulsification pressure was set to 20 MPa. The particle size of the obtained oil-in-water emulsion composition containing licorice polyphenol was measured, and the results of the stability evaluation against acid and heat are summarized in Table 1.

### Example 10

An oil-in-water emulsion composition containing licorice polyphenol was prepared at the blending ratio shown in Table 1. The oil-in-water emulsion composition containing licorice polyphenol was prepared in the same manner as in Example 1 except that licorice hydrophobic extract-containing medium-chain triglyceride solution of Preparation Example 2 (1 part by weight) as an oil phase, decaglycerol monomyristic acid ester (M-7D, 5.5 parts by weight) as polyglycerol fatty acid ester, coenzyme Q10 (manufactured by Kaneka Corporation, 0.1 part by weight), and water (93.4 parts by weight) as an aqueous phase were used. The particle size of the obtained oil-in-water emulsion composition containing licorice polyphenol was measured, and the results of the stability evaluation against acid and heat are summarized in Table 1.

### Example 11

An oil-in-water emulsion composition containing licorice polyphenol was prepared at the blending ratio shown in Table 1. The oil-in-water emulsion composition containing licorice polyphenol was prepared in the same manner as in Example 1 except that licorice hydrophobic extract-containing medium-chain triglyceride solution of Preparation Example 2 (1 part by weight) as an oil phase, decaglycerol monomyristic acid ester (M-7D, 6 parts by weight) as polyglycerol fatty acid ester, coenzyme Q10 (manufactured by Kaneka Corporation, 0.2 parts by weight), and water (92.8 parts by weight) as an aqueous phase were used. The particle size of the obtained oil-in-water emulsion composition containing licorice polyphenol was measured, and the results of the stability evaluation against acid and heat are summarized in Table 1.

### Comparative Example 1

An oil-in-water emulsion composition containing licorice polyphenol was prepared at the blending ratio shown in Table 1. Specifically, licorice hydrophobic extract-containing medium-chain triglyceride solution of Preparation Example 2 (5 parts by weight) as an oil phase and decaglycerol monolauric acid ester (manufactured by SAKAMOTO YAKUHIN KOGYO CO., LTD.; ML-750, 10 parts by weight) were heated to 60° C. and dissolved completely. Similarly, as an aqueous phase, water (85 parts by weight) was heated to 60° C. Then, the aqueous phase was added to the oil phase with stirring, and the mixture was treated 10 times with emulsification pressure of 100 MPa using Nanomizer II (manufactured by Yoshida Machinery Co. Ltd.) to give an oil-in-water emulsion composition containing licorice polyphenol. The particle size of the obtained oil-in-water emulsion composition containing licorice polyphenol was measured, and the results of the stability evaluation against acid and heat are summarized in Table 1.

TABLE 1
Composition (%) of oil-in-water emulsion composition and results

| | | | | | | | | | | | | | Comp. |
|---|---|---|---|---|---|---|---|---|---|---|---|---|---|
| | | Ex. 1 | Ex. 2 | Ex. 3 | Ex. 4 | Ex. 5 | Ex. 6 | Ex. 7 | Ex. 8 | Ex. 9 | Ex. 10 | Ex. 11 | Ex. 1 |
| blending | licorice | 1 | 1 | 5 | 3 | 1 | 1 | 1 | 1 | 1 | 1 | 1 | 5 |
| composition | hydrophobic | | | | | | | | | | | | |
| | extract containing | | | | | | | | | | | | |
| | medium-chain | | | | | | | | | | | | |
| | triglyceride | | | | | | | | | | | | |
| | solution | | | | | | | | | | | | |
| | decaglycerol | | | | | | | | | | | | 10 |
| | monolauric acid | | | | | | | | | | | | |
| | ester (ML-750) | | | | | | | | | | | | |
| | decaglycerol | 5 | | | | 1 | | | 5 | 5 | 5.5 | 6 | |
| | myristic acid | | | | | | | | | | | | |
| | ester (M-7D) | | | | | | | | | | | | |
| | decaglycerol | | 5 | | | | | | | | | | |
| | palmitic acid | | | | | | | | | | | | |
| | ester (P-8DS) | | | | | | | | | | | | |
| | decaglycerol | | | 10 | | | 2 | | | | | | |
| | stearic acid ester | | | | | | | | | | | | |
| | (J-0081) | | | | | | | | | | | | |
| | decaglycerol oleic | | | | 10 | | | | | | | | |
| | acid ester (J-0381) | | | | | | | | | | | | |
| | decaglycerol oleic | | | | | | | 10 | | | | | |
| | acid ester (Q-17S) | | | | | | | | | | | | |
| | Coenzyme Q10 | | | | | | | | | | 0.1 | 0.2 | |
| | water | 94 | 94 | 85 | 87 | 98 | 97 | 89 | 94 | 94 | 93.4 | 92.8 | 85 |
| | total | 100 | 100 | 100 | 100 | 100 | 100 | 100 | 100 | 100 | 100 | 100 | 100 |
| | above-mentioned | 0.24 | 0.24 | 1.20 | 0.72 | 0.24 | 0.24 | 0.24 | 0.24 | 0.24 | 0.24 | 0.24 | 1.20 |
| | polyphenol content | | | | | | | | | | | | |
| | (%) | | | | | | | | | | | | |
| | above-mentioned | 0.03 | 0.03 | 0.15 | 0.09 | 0.03 | 0.03 | 0.03 | 0.03 | 0.03 | 0.03 | 0.03 | 0.15 |
| | glabridin content | | | | | | | | | | | | |
| | (%) | | | | | | | | | | | | |
| | median size (nm) | 24.3 | 18.8 | 28.8 | 155.9 | 28.3 | 34.3 | 25.6 | 38.8 | 115.9 | 35.1 | 56.1 | 13.6 |
| | of obtained oil- | | | | | | | | | | | | |
| | in-water emulsion | | | | | | | | | | | | |
| | composition | | | | | | | | | | | | |
| stability | addition of water | 25.7 | 38.6 | 44.7 | 28.7 | 41.3 | 52.1 | 26.4 | 61.5 | 106.2 | 35.8 | 56 | 59.5 |
| evaluation | (nm) | | | | | | | | | | | | |
| median size | same as above | 18.5 | 26.9 | 45.9 | 36.7 | 40.8 | 48.5 | 28.5 | 61.6 | 108 | 37.6 | 61.5 | 53.5 |
| after each | 75° C. × | | | | | | | | | | | | |
| treatment | 15 min (nm) | | | | | | | | | | | | |
| | pH 3 water addition | 28.0 | 53.7 | 46.1 | 34.9 | 48.1 | 52.7 | 29.1 | 63.7 | 98.7 | 37.2 | 52.1 | 84.7 |
| | (nm) | | | | | | | | | | | | |
| | same as above | 15.0 | 53.6 | 45.9 | 77.8 | 71.3 | 102.4 | 54.5 | 113.5 | 114.4 | 29.6 | 69.6 | 254.3 |
| | 75° C. × | | | | | | | | | | | | |
| | 15 min (nm) | | | | | | | | | | | | |

### Example 12

An oil-in-water emulsion composition containing licorice polyphenol was prepared at the blending ratio shown in Table 2. The oil-in-water emulsion composition containing licorice polyphenol was prepared in the same manner as in Example 1 except that licorice hydrophobic extract-containing medium-chain triglyceride solution of Preparation Example 2 (1 part by weight) as an oil phase, decaglycerol monomyristic acid ester (M-7D, 5.5 parts by weight) as polyglycerol fatty acid ester, reduced coenzyme Q10 (manufactured by Kaneka Corporation, 0.1 part by weight), and water (93.4 parts by weight) as an aqueous phase were used. The particle size of the obtained oil-in-water emulsion composition containing licorice polyphenol was measured, and the results of the stability evaluation against acid and heat are summarized in Table 2.

### Example 13

An oil-in-water emulsion composition containing licorice polyphenol was prepared at the blending ratio shown in Table 2. The oil-in-water emulsion composition containing licorice polyphenol was prepared in the same manner as in Example 1 except that licorice hydrophobic extract-containing medium-chain triglyceride solution of Preparation Example 2 (1 part by weight) as an oil phase, decaglycerol monomyristic acid ester (M-7D, 6 parts by weight) as polyglycerol fatty acid ester, reduced coenzyme Q10 (manufactured by Kaneka Corporation, 0.2 parts by weight), and water (92.8 parts by weight) as an aqueous phase were used. The particle size of the obtained oil-in-water emulsion composition containing licorice polyphenol was measured, and the results of the stability evaluation against acid and heat are summarized in Table 2.

### Example 14

An oil-in-water emulsion composition containing licorice polyphenol was prepared at the blending ratio shown in Table 2. The oil-in-water emulsion composition containing licorice polyphenol was prepared in the same manner as in Example 1 except that licorice hydrophobic extract-containing medium-chain triglyceride solution of Preparation Example 2 (5 parts by weight) as an oil phase, decaglycerol monooleic acid ester (J-0381, 10 parts by weight) as polyglycerol fatty acid ester, glycerol (manufactured by SAKAMOTO YAKUHIN KOGYO CO., LTD., 76.5 parts by weight) and water (8.5 parts by weight) as an aqueous phase were used. The particle size of the obtained oil-in-water emulsion composition containing licorice polyphenol was measured, and the results of the stability evaluation against acid and heat are summarized in Table 2.

### Example 15

An oil-in-water emulsion composition containing licorice polyphenol was prepared at the blending ratio shown in Table 2. The oil-in-water emulsion composition containing licorice polyphenol was prepared in the same manner as in Example 1 except that licorice hydrophobic extract-containing medium-chain triglyceride solution of Preparation Example 2 (5 parts by weight) as an oil phase, decaglycerol monooleic acid ester (J-0381, 10 parts by weight) as polyglycerol fatty acid ester, liquid sugar syrup (manufactured by Showa Sangyo Co., Ltd.; New Fructo R-30, 67.55 parts by weight) and water (17.45 parts by weight) as an aqueous phase were used. The particle size of the obtained oil-in-water emulsion composition containing licorice polyphenol was measured, and the results of the stability evaluation against acid and heat are summarized in Table 2.

### Example 16

An oil-in-water emulsion composition containing licorice polyphenol was prepared at the blending ratio shown in Table 2. The oil-in-water emulsion composition containing licorice polyphenol was prepared in the same manner as in Example 1 except that licorice hydrophobic extract-containing medium-chain triglyceride solution of Preparation Example 2 (5 parts by weight) as an oil phase, decaglycerol monooleic acid ester (J-0381, 10 parts by weight) as polyglycerol fatty acid ester, sorbitol (manufactured by UENO FINE CHEMICALS INDUSTRY. LTD.; Sorbitol-UENO, 72.86 parts by weight), and water (12.14 parts by weight) as an aqueous phase were used. The particle size of the obtained oil-in-water emulsion composition containing licorice polyphenol was measured, and the results of the stability evaluation against acid and heat are summarized in Table 2.

### Example 17

An oil-in-water emulsion composition containing licorice polyphenol was prepared at the blending ratio shown in Table 2. The oil-in-water emulsion composition containing licorice polyphenol was prepared in the same manner as in Example 1 except that licorice hydrophobic extract-containing medium-chain triglyceride solution of Preparation Example 2 (10 parts by weight) as an oil phase, decaglycerol monooleic acid ester (J-0381, 20 parts by weight) as polyglycerol fatty acid ester, glycerol (56 parts by weight), and water (14 parts by weight) as an aqueous phase were used. The particle size of the obtained oil-in-water emulsion composition containing licorice polyphenol was measured, and the results of the stability evaluation against acid and heat are summarized in Table 2.

### Example 18

An oil-in-water emulsion composition containing licorice polyphenol was prepared at the blending ratio shown in Table 2. Licorice hydrophobic extract-containing medium-chain triglyceride solution of Preparation Example 2 (5 parts by weight) as an oil phase and decaglycerol monooleic acid ester (J-0381, 10 parts by weight) as polyglycerol fatty acid ester were heated to 60° C. and dissolved completely. Similarly, glycerol (68 parts by weight) and water (17 parts by weight) as an aqueous phase were heated to 60° C. Then, the aqueous phase was added to the oil phase and the mixture was stirred with a chemistirrer. The particle size of the obtained oil-in-water emulsion composition containing licorice polyphenol was measured, and the results of the stability evaluation against acid and heat are summarized in Table 2.

TABLE 2
Composition (%) of oil-in-water emulsion compositions and results

| | | Ex. 12 | Ex. 13 | Ex. 14 | Ex. 15 | Ex. 16 | Ex. 17 | Ex. 18 |
|---|---|---|---|---|---|---|---|---|
| blending | licorice hydrophobic extract | 1 | 1 | 5 | 5 | 5 | 10 | 5 |
| composition | containing medium-chain triglyceride | | | | | | | |
| | solution | | | | | | | |
| | decaglycerol myristic acid ester | 5.5 | 6 | | | | | |
| | (M-7D) | | | | | | | |
| | decaglycerol oleic acid ester | | | 10 | 10 | 10 | 20 | 10 |
| | (J-0381) | | | | | | | |
| | reduced coenzyme Q10 | 0.1 | 0.2 | | | | | |
| | glycerol | | | 76.5 | | | 56 | 68 |
| | liquid sugar syrup | | | | 67.55 | | | |
| | sorbitol | | | | | 72.86 | | |
| | water | 93.4 | 92.8 | 8.5 | 17.45 | 12.14 | 14 | 17 |
| | total | 100 | 100 | 100 | 100 | 100 | 100 | 100 |
| | above-mentioned polyphenol content | 0.24 | 0.24 | 1.20 | 1.20 | 1.20 | 2.40 | 1.20 |
| | (%) | | | | | | | |
| | above-mentioned glabridin content | 0.03 | 0.03 | 0.15 | 0.15 | 0.15 | 0.30 | 0.15 |
| | (%) | | | | | | | |
| | median size (nm) of obtained oil-in- | 35.1 | 48.3 | 24.8 | 48.2 | 28.3 | 70.7 | 47.1 |
| | water emulsion composition | | | | | | | |
| stability | water added (nm) | 35.5 | 48.1 | 33.5 | 44.2 | 35.9 | 102.4 | 51.0 |
| evaluation | same as above | 35.9 | 40.2 | 31.5 | 42.3 | 37.4 | 101.1 | 50.5 |
| median size | 75° C. × 15 min (nm) | | | | | | | |
| after each | pH 3 water addition (nm) | 31.6 | 43.3 | 36.2 | 50.5 | 37.3 | 91.4 | 48.7 |
| treatment | same as above | 38.3 | 52.5 | 77.5 | 114.3 | 104.3 | 130.7 | 116.6 |
| | 75° C. × 15 min (nm) | | | | | | | |

From the results of Table 1 and Table 2, it is clear that the oil-in-water emulsion composition containing licorice polyphenol of the present invention is superior in transparency, and is an emulsion highly stable against acid and heat.

### Comparative Example 2

Decaglycerol monolauric acid ester (90 parts by weight) was added to the licorice hydrophobic extract-containing ethanol solution (50 parts by weight, containing licorice hydrophobic extract (10 parts by weight)) of Preparation Example 1. The mixture was dissolved in ethanol, and ethanol was removed by concentration under reduced pressure to give a composition containing licorice hydrophobic extract. Water parts by weight) was added to the composition containing licorice hydrophobic extract (15 parts by weight) to give an aqueous solution containing licorice polyphenol. An aqueous citric acid solution (50 parts by weight, pH 3) was added to the aqueous solution containing licorice polyphenol (1 part by weight), and the median size was measured using LB-550 after the addition and after heating the mixture at 75° C. for 15 min. The results are summarized in Table 3.

TABLE 3

| | Comparative Example 2 |
|---|---|
| aqueous solution containing licorice | 1.20 |
| polyphenol | |
| polyphenol content (%) | |
| aqueous solution containing licorice | 0.15 |
| polyphenol | |
| glabridin content (%) | |
| stability evaluation | 66.5 |
| median size (nm) after pH 3 water | |
| addition | |
| same as above, | 139.6 |
| after treatment 75° C. × 15 min (nm) | |

### Comparative Example 3

An oil-in-water emulsion composition containing licorice polyphenol was prepared at the blending ratio shown in Table 4. Specifically, licorice hydrophobic extract-containing medium-chain triglyceride solution of Preparation Example 2 (1 part by weight) as an oil phase and sucrose stearic acid ester (manufactured by DAI-ICHI KOGYO SEIYAKU CO., LTD.; DK ester SS, 5 parts by weight) as an emulsifier were heated to 60° C. and dissolved completely. Similarly, as an aqueous phase, water (94 parts by weight) was heated to 60° C. Then, the aqueous phase was added to the oil phase with stirring, and the mixture was treated 10 times at a emulsification pressure of 100 MPa using Nanomizer II (manufactured by Yoshida Machinery Co., Ltd.) to give an oil-in-water emulsion composition containing licorice polyphenol. The particle size of the obtained oil-in-water emulsion composition containing licorice polyphenol was measured (Table 4). Subsequently, the stability evaluation of the oil-in-water emulsion composition against acid and heat was tried. However, when water and citric acid aqueous solution were added, the emulsification became broken and separated, and the emulsion particle size could not be measured.

### Comparative Example 4

An oil-in-water emulsion composition containing licorice polyphenol was prepared at the blending ratio shown in Table 4. The oil-in-water emulsion composition containing licorice polyphenol was prepared in the same manner as in Comparative Example 3 except that licorice hydrophobic extract-containing medium-chain triglyceride solution of Preparation Example 2 (3 parts by weight) as an oil phase, sucrose stearic acid ester (manufactured by DAI-ICHI KOGYO SEIYAKU CO., LTD.; DK ester SS, 10 parts by weight) as an emulsifier, and water (87 parts by weight) as an aqueous phase were used, and the particle size of the obtained oil-in-water emulsion composition containing licorice polyphenol was measured (Table 4). Subsequently, the stability evaluation of the oil-in-water emulsion composition against acid and heat was tried. However, when water and citric acid aqueous solution were added, the emulsification became broken and separated as in Comparative Example 3, and the emulsion particle size could not be measured.

### Comparative Example 5

An oil-in-water emulsion composition containing licorice polyphenol was prepared at the blending ratio shown in Table 4. The oil-in-water emulsion composition containing licorice polyphenol was prepared in the same manner as in Comparative Example 3 except that licorice hydrophobic extract-containing medium-chain triglyceride solution of Preparation Example 2 (5 parts by weight) as an oil phase, sucrose stearic acid ester (manufactured by DAI-ICHI KOGYO SEIYAKU CO., LTD.; DK ester SS, 10 parts by weight) as an emulsifier, and water (85 parts by weight) as an aqueous phase were used, and the particle size of the obtained oil-in-water emulsion composition containing licorice polyphenol was measured (Table 4). Subsequently, the stability evaluation of the oil-in-water emulsion composition against acid and heat was tried. However, when water and citric acid aqueous solution were added, the emulsification became broken and separated as in Comparative Example 3, and the emulsion particle size could not be measured.

TABLE 4

| | | Comp. | Comp. | Comp. |
|---|---|---|---|---|
| | | Ex. 3 | Ex. 4 | Ex. 5 |
| blending | licorice hydrophobic | 1 | 3 | 5 |
| composition | extract-containing | | | |
| | medium-chain | | | |
| | triglyceride solution | | | |
| | sucrose stearic acid | 5 | 10 | 10 |
| | ester (DK ester SS) | | | |
| | water | 94 | 87 | 85 |
| | total | 100 | 100 | 100 |
| | above-mentioned | 0.24 | 0.72 | 1.20 |
| | polyphenol content (%) | | | |
| | above-mentioned | 0.03 | 0.09 | 0.15 |
| | glabridin content (%) | | | |
| | median size (nm) of | 1247.7 | 954.3 | 1329.4 |
| | obtained oil-in-water | | | |
| | emulsion composition | | | |

### Example 19

A dry powder of an oil-in-water emulsion composition containing licorice polyphenol was prepared by the following formulation. Licorice hydrophobic extract-containing medium-chain triglyceride solution of Preparation Example 2 (2 parts by weight) as an oil phase, decaglycerol monomyristic acid ester (M-7D, 3 parts by weight) as an emulsifier, gum arabic (manufactured by Colloides Naturels Japan. Inc.; INSTANTGUM AA, 15 parts by weight) and water (180 parts by weight) were added and the mixture was stirred. Furthermore, the mixture was homogenized at 10000 rpm for 10 min using a homogenizer (manufactured by KINEMATICA). Water was removed by spray drying (manufactured by Nihon BUCHI K.K.) to give a dry powder of an oil-in-water emulsion composition containing licorice polyphenol. The licorice polyphenol content of the powder was 2.4%, and the glabridin content of the powder was 0.3%. In addition, an aqueous solution wherein the prepared powder was dissolved to a licorice polyphenol content of 0.024% and a glabridin content of 0.003% was transparent, and the particle size thereof was 160 nm.

### Example 20

A dry powder of an oil-in-water emulsion composition containing licorice polyphenol was prepared by the following formulation. Licorice hydrophobic extract-containing medium-chain triglyceride solution of Preparation Example 2 (6 parts by weight) as an oil phase, decaglycerol monomyristic acid ester (manufactured by Mitsubishi-Kagaku Foods Corporation; M-7D, 1 part by weight) as an emulsifier, gum arabic (manufactured by Colloides Naturels Japan. Inc.; INSTANTGUM AA, 12 parts by weight), erythritol (manufactured by Mitsubishi-Kagaku Foods Corporation, 1 part by weight) and water (180 parts by weight) were added and the mixture was stirred. Furthermore, the mixture was homogenized at 10000 rpm for 10 min using a homogenizer (manufactured by KINEMATICA). Water was removed by spray drying (manufactured by Nihon BUCHI K.K.) to give a dry powder of an oil-in-water emulsion composition containing licorice polyphenol. The licorice polyphenol content of the powder was 7.2%, and the glabridin content of the powder was 0.9%. In addition, an aqueous solution wherein the prepared powder was dissolved to a glabridin content of 0.003% was transparent.

### Example 21

### Production of Drink Preparation

As a licorice polyphenol-containing food, a beverage containing licorice polyphenol and coenzyme Q10 was prepared according to the following formulation.

<formulation>

| | | |
|---|---|---|
| sugar | 10 | parts by weight |
| citric acid (anhydrous) | 0.2 | part by weight |
| trisodium citrate | 0.02 | part by weight |
| water | 87.28 | parts by weight |
| oil-in-water emulsion composition | 2.4 | parts by weight |
| containing licorice polyphenol | | |
| prepared in Example 11 | | |
| orange flavor | 0.1 | part by weight |

### <Preparation Method>

Sugar, citric acid and trisodium citrate were dissolved in water, and an oil-in-water emulsion composition containing licorice polyphenol prepared in Example 11 and orange flavor were added thereto. The mixture was sterilized in a water bath at an inside temperature of 70° C. for 20 min and cooled with water to give a beverage containing licorice polyphenol and coenzyme Q10.

This application is based on a patent application Nos. 2006-048362 and 2006-198909 filed in Japan, the contents of which are incorporated in full herein by this reference.

1. A licorice polyphenol-containing oil-in-water emulsion composition comprising medium-chain triglyceride, a licorice hydrophobic extract comprising, as a main component, licorice polyphenol in a weight ratio relative to the medium-chain triglyceride of 1-50%, and a polyglycerol fatty acid ester comprised of a fatty acid residue having a carbon number of not less than 14.

2. The oil-in-water emulsion composition of claim 1, wherein the weight ratio of the total weight of the licorice hydrophobic extract and the medium-chain triglyceride to the polyglycerol fatty acid ester is within the range of 10:1-1:10.

3. The oil-in-water emulsion composition of claim 1, wherein the degree of polymerization of polyglycerol in the polyglycerol fatty acid ester is not less than 6.

4. The oil-in-water emulsion composition of claim 1, wherein the degree of polymerization of polyglycerol in the polyglycerol fatty acid ester is 10, and the fatty acid residue is at least one kind selected from the group consisting of myristic acid, palmitic acid, stearic acid and oleic acid.

5. The oil-in-water emulsion composition of claim 1, further comprising coenzyme Q10.

6. The oil-in-water emulsion composition of claim 1, having a median size of emulsion particles of not more than 100 nm.

7. The oil-in-water emulsion composition of claim 6, which is obtained by a homogenization pressure treatment at not less than 50 MPa.

8. A dry powder obtained by adding an excipient to the oil-in-water emulsion composition of claim 1, and removing water from the mixture.

9. A food, drink, pharmaceutical product, quasi-drug, cosmetic or feed for animal, comprising the oil-in-water emulsion composition of claim 1.

10. The drink of claim 9, wherein the content of licorice polyphenol is 10-200 mg per a package unit.

11. The drink of claim 9, wherein the content of glabridin is 1-20 mg per a package unit.

12. A food, drink, pharmaceutical product, quasi-drug, cosmetic or feed for animal, comprising the dry powder of claim 8.

13. The drink of claim 12, wherein the content of licorice polyphenol is 10-200 mg per a package unit.

14. The drink of claim 12, wherein the content of glabridin is 1-20 mg per a package unit.
